(19) United States
(12) Patent Application Publication
(10) Pub. No.: US 2012/0065496 A1
(43) Pub. Date: Mar. 15, 2012

(54) APPARATUS FOR IMAGING A BODY PART

(75) Inventors: Mathew David Frederick Stratton, Stroud (GB); Hugo George Derrick, Stroud (GB)

(73) Assignee: RENISHAW (IRELAND) LIMITED, Swords (IE)

(21) Appl. No.: 13/320,942

(22) PCT Filed: May 20, 2010
(86) PCT No.: PCT/GB2010/001013

§ 371 Date: Nov. 17, 2011

(30) Foreign Application Priority Data
May 21, 2009 (GB) 0908784.2

(51) Int. Cl. A61B 5/055

(52) U.S. Cl. 600/414; 600/421

(57) ABSTRACT

Apparatus for imaging a body part of a subject, for example using MRI, is described. The apparatus includes a housing for at least partially surrounding a body part (e.g. a head) and a first fiducial marker assembly retained at least partially within the housing that includes one or more fiducial markers and a datum feature. The position of the datum feature is fixed relative to the one or more fiducial markers. The first fiducial marker assembly is moveable with respect to the housing and the datum feature is accessible from outside of the housing.

The present invention relates to apparatus for imaging a body part of a subject, and in particular to apparatus for magnetic resonance imaging of the head of a subject.

In stereotactic neurosurgery, a neurosurgeon will often have to insert instruments to targets within the brain with millimetre accuracy. In particular, the success of stereotactic functional neurosurgery is highly dependent on the accuracy with which neurosurgical instruments, such as electrodes or catheters, can be guided to a predetermined target site of the brain. Stereotactic head frames for use in precision guided neurosurgery are known. For example, the Leksell (Registered Trademark) frame produced by Elekta AB, Sweden includes a base ring attachable to the base of the skull. The base ring can be locked to an operating table to immobilise the head and various neurosurgical instruments can also be secured to the base ring. Once the base ring is attached to the subject's head and has been imaged it acts as a platform of known position relative to the head. The base ring can thus be used as a reference position for acquired images and a platform from which instruments can be stereotactically guided to the required target site or sites in the brain.

It has also been proposed previously to mount MRI coils and fiducial markers to the base ring of a Leksell type frame to allow the position of targets found in MRI images to be determined relative to the base ring. For example, WO2006/134357 describes apparatus in which MRI imaging coils and fiducial markers can be attached directly to such a base ring.

The above described type of frames have been found, however, to have several disadvantages. In particular, the base ring of such frames can limit the size and position of RF coils that can used for MRI of the head thereby limiting the imaging resolution, especially for regions deep within the brain.

According to a first aspect of the present invention, apparatus for imaging a body part of a subject is provided that comprises a housing for at least partially surrounding a body part, and a first fiducial marker assembly retained at least partially within the housing that comprises one or more fiducial markers and a datum feature, the position of the datum feature being fixed relative to the one or more fiducial markers, wherein the first fiducial marker assembly is moveable with respect to the housing and the datum feature is accessible from outside of the housing.

The present invention thus provides apparatus that is suitable for use with various medical imaging techniques, such as MRI, ultrasound or CT scanning. The apparatus includes a housing that at least partially surrounds a body part (e.g. the head) of a subject and a first fiducial marker assembly that comprises one or more fiducial markers that have a fixed position relative to a datum feature of that fiducial marker assembly. The first fiducial marker assembly is retained at least partially within the housing but is moveable with respect to that housing. The first fiducial marker assembly is thus free to move within housing but at least part of it is retained therein; i.e. the first fiducial marker assembly is at least partially retained within, but it is not fully constrained by, the housing. In this manner, the first fiducial marker assembly is sufficiently mechanically isolated from the housing such that any distortions of the housing that may occur in use are not transmitted to the first fiducial marker assembly thereby ensuring the positions of the one or more fiducial markers are accurately maintained relative to the datum feature. In addition, the datum feature of the first fiducial marker assembly is accessible from outside of the housing thereby allowing the datum feature to be mated with a complementary datum feature provided on, for example, a fixture that is attached to the body part to be imaged.

The apparatus of the present invention thus allows a fiducial marker assembly, and in particular one or more fiducial markers, to be repeatably placed in a predetermined position relative to an associated fixture. In particular, any distortions of the housing that may occur during use will have no substantial effect on the position of the fiducial markers relative to the complementary datum feature of an associated fixture. In this manner, it can be ensured that the fiducial markers that appear in any images acquired using the apparatus are located at a known position relative to the complementary datum feature of an associated fixture to a higher level of accuracy than is typically possible using prior art apparatus of the type described above. The present invention thus provides an improvement to the accuracy and reliability of stereotactic neurosurgery procedures thereby improving the clinical outcome of such procedures.

The housing of the apparatus preferable defines an internal cavity. For example, the housing may comprise a plastic casing or shell having an internal space in which various components can be located. It should also be noted that the internal cavity of the housing is used herein to refer to a space which is normally inaccessible during use of the apparatus. In particular, the internal cavity defined by the housing should not be confused with the body part imaging space that the housing can at least partially surround.

Although apparatus of the present invention is suitable for use with any medical imaging technique (e.g. CT, ultrasound etc), the apparatus is preferably configured for use with MRI apparatus. Advantageously, one or more RF coils for magnetic resonance imaging are provided within the internal cavity of the housing. For example, the housing may house rigid or flexible RF coil assemblies. Electronic circuitry associated with such RF coil assemblies may also be located within the housing. The housing may thus also act as an electrically protective cover that prevents users from accessing, during normal use, the voltage signals applied to the RF coils. Similarly, the one or more fiducial markers may be visible to any one or more imaging technique. Advantageously, the one or more fiducial markers can be imaged using magnetic resonance imaging. The one or more fiducial markers conveniently provide positional information in at least one, at least two, or three, mutually orthogonal axes.

As outlined above, the datum feature of the first fiducial marker assembly can be accessed from outside of the housing. Advantageously, the housing comprises an aperture that provides access to the datum feature. Conveniently, the portion of the first fiducial marker assembly that comprises the datum feature protrudes from the housing through the aperture. In other words, the datum feature may be arranged to protrude through an aperture and stand proud of the surface of the housing. In this manner, the datum feature can be easily mated with a complementary datum feature. A flexible connection or linkage may also be provided to loosely secure the first fiducial marker assembly in place relative to the housing. For example, a spring bias may be provided to allow the first fiducial marker assembly to float within the housing.

The housing may be formed from a plurality of housing parts. The plurality of housing parts may be configured to define an imaging space in which the required body part can be placed. Advantageously, the housing comprises a first housing part and a second housing part. Conveniently, the first and second housing parts can be moved into a closed position that defines an imaging space. Advantageously, a human head can be located in such an imaging space. In other words, inner faces of the first and second housing parts may be shaped to surround parts of a human head. Advantageously, the first and second housing parts can also be moved into an open position in which a human head can be located between the first and second housing parts such that, when the first and second housing parts are moved into the closed position, the housing substantially surrounds the human head.

The housing parts may be connected to one another in a variety of ways.

Advantageously, the first housing part is attached to the second housing part via a pivot joint. The first and second housing parts may then be pivoted into the closed position. Advantageously, the first and second housing parts pivot together from opposite sides of the head. If such a pivot joint is provided, it may also be used to route cables (e.g. connected to RF coils located in the housing) to and/or from an associated interface or imaging machine. In other words, one or more electrical cables are preferably routed to and/or from the first and second housing parts through the pivot joint. This arrangement reduces the need for trailing wires and also protects such wires from being accidentally damaged.

In addition to the first fiducial marker assembly, the apparatus may comprise one or more additional fiducial marker assemblies. Each fiducial marker assembly may comprise one or more fiducial markers and a datum feature having a fixed position relative to its one or more fiducial markers. Each fiducial marker assembly may comprise a datum feature accessible from outside of the housing and/or may be moveable relative to the housing. Preferably, the apparatus comprises a second fiducial marker assembly comprising one or more fiducial markers located within the housing that have a fixed position relative to a datum feature that is accessible from outside of the housing. The second fiducial marker assembly may be mechanically earthed (e.g. secured to the housing). Preferably, the second fiducial marker assembly is moveable relative to the housing. Advantageously, the housing comprises two parts as described above in which case the first housing part contains the first fiducial marker assembly and the second housing part contains the second fiducial marker assembly.

The apparatus is conveniently arranged to receive a head to which a head clamp is attached. Preferably, the associated head clamp comprises a pair of complementary datum features. Advantageously, the datum features of the first and second fiducial marker assemblies described above are brought into contact with the complementary datum features provided on the head clamp when the first and second parts of the housing are moved into the closed position. In this manner, the act of closing the first and second housing parts acts to urge the datum features of the first and second fiducial marker assemblies into contact with complementary datum features provide on the head clamp. In this manner, the fiducial markers of the first and second fiducial marker assemblies are located in known positions relative to the datum features of the head clamp as the housing is closed around that clamp.

As mentioned above, the apparatus may receive a head clamp secured to the head of a subject. If other body parts are to be imaged, they may also have a suitable fixture attached thereto. Preferably, the apparatus comprises a clamp mechanism for releasably retaining a fixture that is attached to the body part of a subject. For example, the fixture may comprise a head clamp. The clamp mechanism may be arranged to hold the fixture in an approximate or pseudo-repeatable position within the apparatus. The fixture is conveniently clamped to the apparatus in a way that still allows the datum feature of the fiducial marker assembly or assemblies to engage complementary datum feature(s) of the fixture. For example, the clamp mechanism may secure a head clamp in place prior to a two-part housing being moved into a closed position as described above in which complementary datum features of the head clamp and the fiducial marker assembly are brought in engagement. The complementary datum features of the head clamp and the fiducial marker assembly may engage directly with one another and/or at least one intermediate part may be provided through which they are attached together.

Each datum feature preferably provides a positional reference point on or in a fixed positional relationship to the fiducial marker assembly. A datum feature may also provide one or more of the fiducial markers; e.g. a combined fiducial marker and datum feature may be provided. Each datum feature may be a marking (e.g. a visible or MRI visible marking) or a physical feature or set of features. Preferably, the datum feature comprises a kinematic datum feature. A datum feature may comprise a plurality of sub-features (e.g. one or more balls and/or grooves). The datum feature of the apparatus of the present invention may thus comprise one part of a kinematic joint that defines a unique datum point or position. Such a kinematic datum feature may be arranged so that it uniquely constrains an associated, complementary, kinematic datum feature in each of the 6 degrees of freedom. For example, a first datum feature may comprise three v-grooves radially spaced apart from one another by 120° and extending along directions that intersect at a common point. A second, complementary, datum feature may then comprise three balls (or at least partly spherical features) spaced in a circle and separated from each other by 120°. The first and second datum features thus provide a highly repeatable kinematic link in which there is only one constraint on each degree of freedom of movement. In this example, either the first or second datum feature may be provided on the apparatus.

The present invention also extends to a kit that comprises the apparatus for imaging a body part of a subject described above and a fixture that is attachable to the body part. The fixture preferably comprises at least one datum feature that is complementary to the datum feature of the apparatus thereby allowing the first fiducial marker assembly to be repeatably located in a predetermined position relative to the fixture.

The fixture may comprise a head clamp. Advantageously, the head clamp comprises a member for at least partially encircling the head of a subject. The member may be a c-shaped member. The head clamp may also comprise at least first and second skull attachment portions for attaching the member to the head of a subject. For example, first and second skull attachment portions may be provided at first and second ends of a c-shaped member. Preferably, the head clamp comprises a position setter that allows the member to be moved between at least two repeatable relative positions. The position setter may comprise a position encoder (e.g. a rotary encoder) or positional markings that measure, or allow to be measured, an angle between the member and a part of the head clamp that has an invariant position relative to the skull to which it is attached. Advantageously, the position setter comprises an indexing mechanism that allows, when the head clamp is attached to a head, the member to be indexed between two or more repeatable relative positions. Such a head clamp is described in more detail in Applicant's co-pending PCT patent application, the contents of which are hereby incorporated by reference, that has the same filing date as the present application and claims priority from UK patent application 0908787.

Conveniently, the head clamp comprises a member for at least partially encircling the head of a subject, and at least first and second skull attachment portions for attaching the member to the head of a subject. For example, the head clamp may comprise a c-shaped member having first and second skull attachment portions provided at first and second ends thereof. Preferably, the member comprises at least one datum feature that is located substantially on a neutral axis of distortion of the member. Such a head clamp is described in more detail in Applicant's co-pending PCT patent application, the contents of which are hereby incorporated by reference, that has the same filing date as the present application and claims priority from UK patent application 0908785.

Also described herein is apparatus for imaging a head, the apparatus comprising a housing for at least partially surrounding the head, wherein the housing comprises first and second housing parts that pivot together from opposite sides of the head to close around the head. Each housing part may comprise a fiducial marker assembly retained at least partially within the housing. Each fiducial marker assembly may comprise one or more fiducial markers and a datum feature, the position of the datum feature being fixed relative to the one or more fiducial markers. Each fiducial marker assembly may be moveable with respect to the housing and have a datum feature that is accessible from outside of the housing.

A fiducial assembly may also be provided that comprises at least one fiducial marker (e.g. at least one MRI visible fiducial marker) and a datum feature having a fixed position relative to the at least one fiducial marker. The datum feature may be combined with one or more fiducial markers. The datum feature preferably allow associated apparatus to be located in a known position relative to the assembly. The fiducial assembly may include means for attachment to the skull of a subject. For example, such means may comprise a clamp (e.g. a c-clamp) for securing the fiducial assembly to a skull.

The invention will now be described, by way of example only, with reference to the accompanying drawings in which;

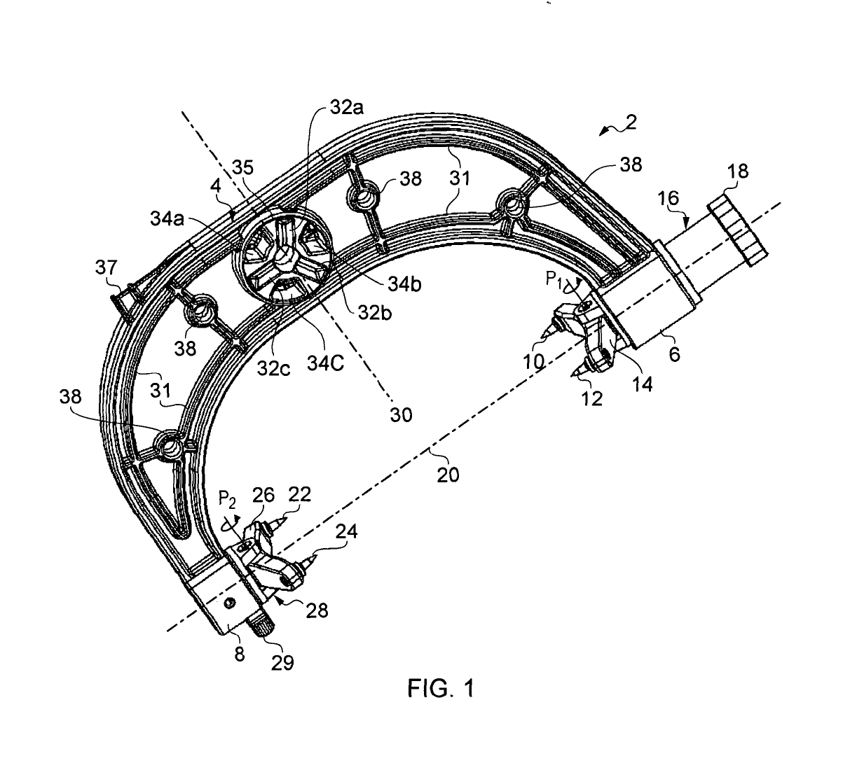

FIG. 1 illustrates a head clamp of the present invention,

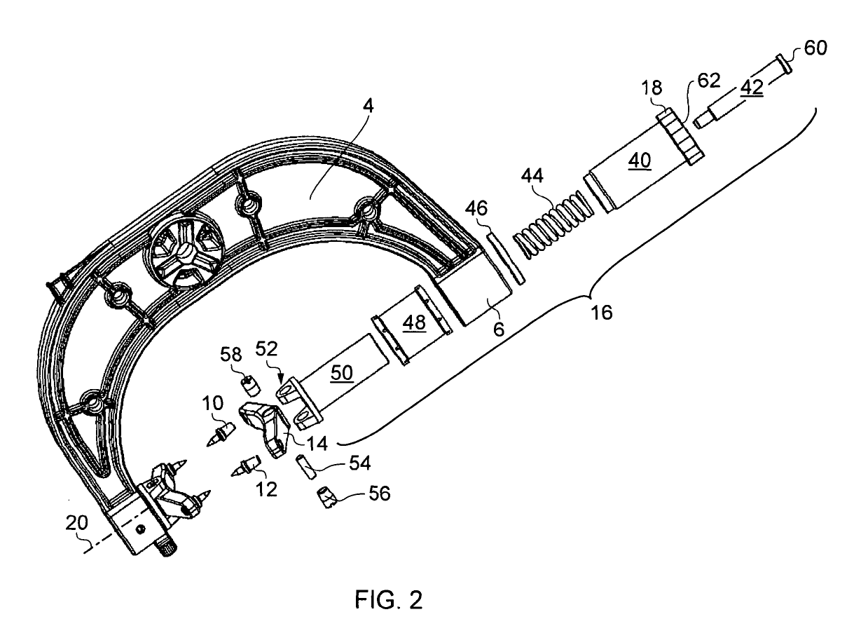

FIG. 2 shows an exploded view of the force control mechanism of the head clamp of FIG. 1,

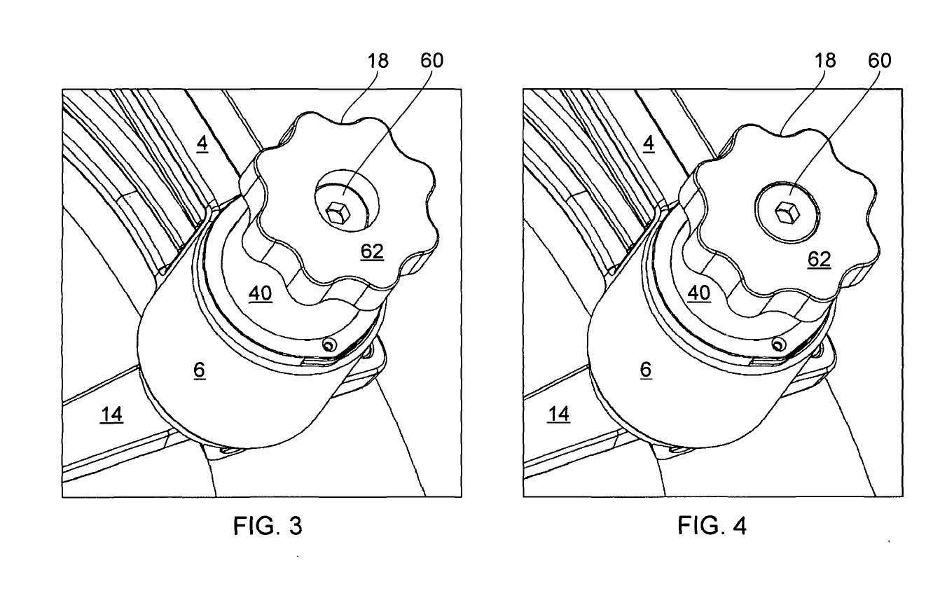

FIG. 3 is a view of the force control mechanism prior to the required force being applied,

FIG. 4 is a view of the force control mechanism when the required force is being applied,

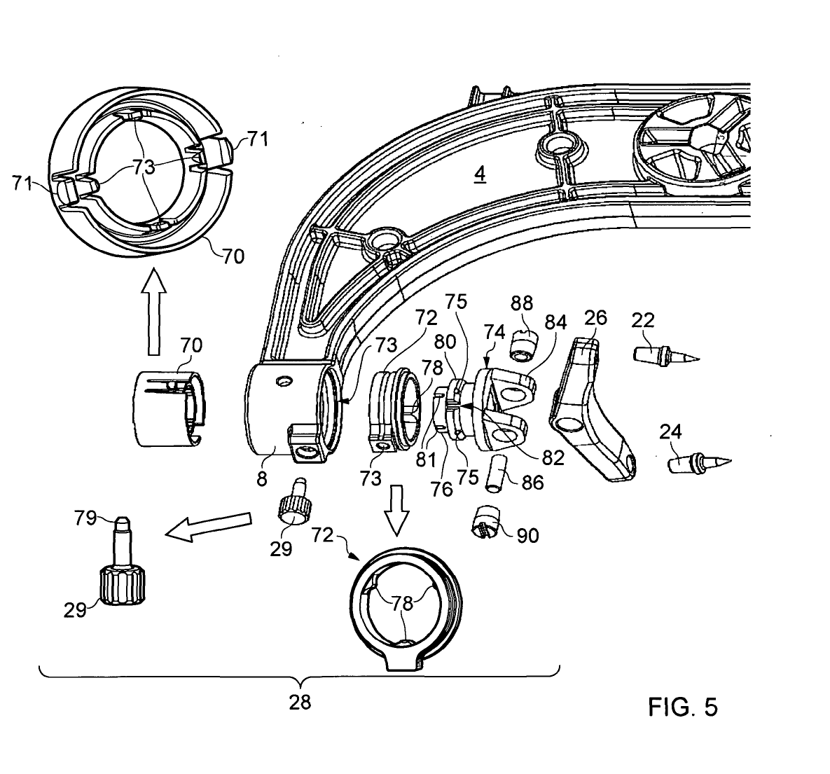

FIG. 5 is an exploded view of the indexing mechanism of the head clamp of FIG. 1,

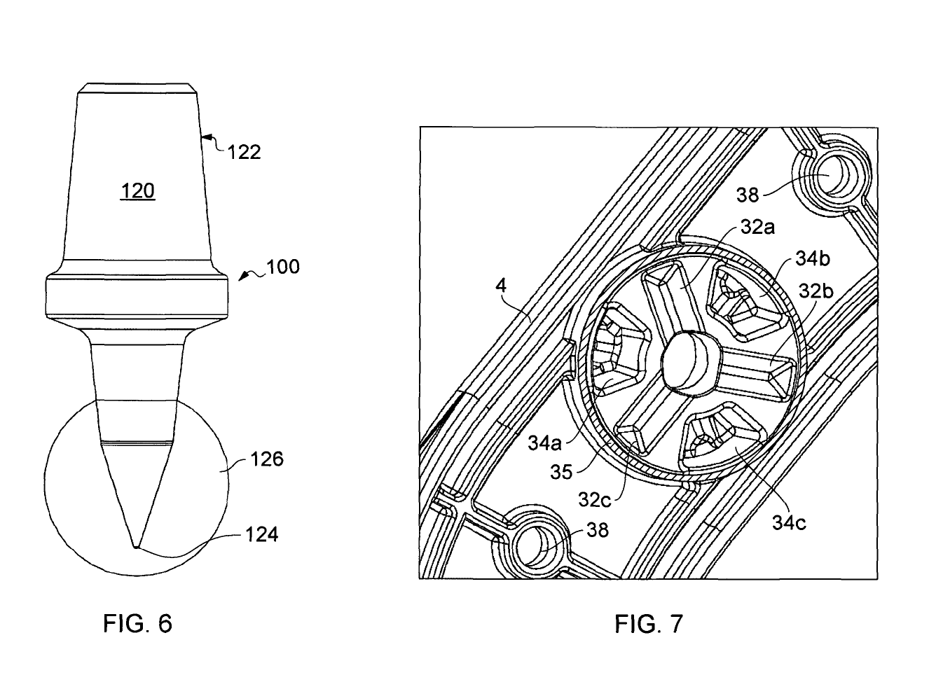

FIG. 6 illustrates a rubber coated attachment pin,

FIG. 7 shows the kinematic datum feature of the head clamp in more detail,

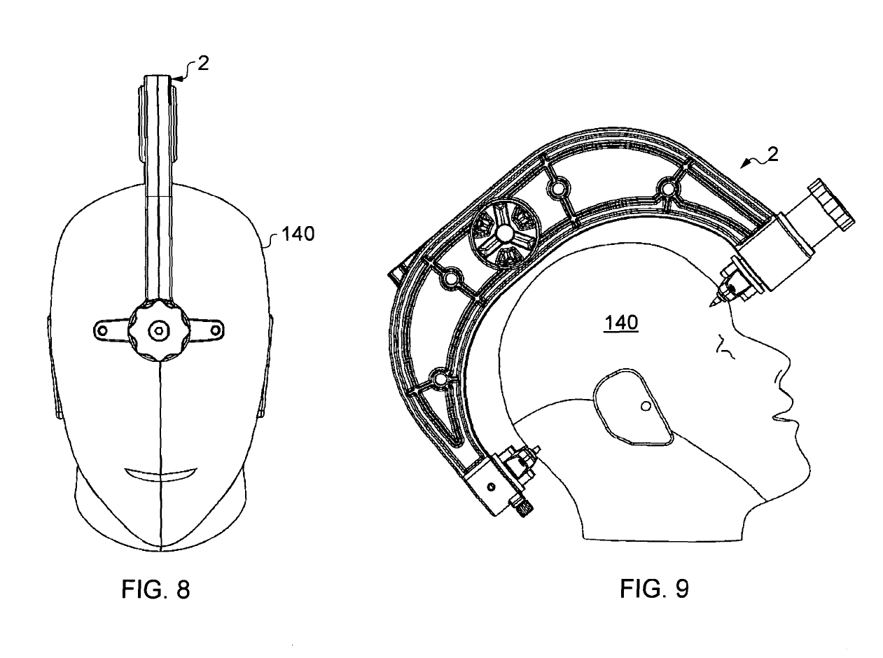

FIG. 8 shows a frontal view of the head clamp when attached to a head and indexed into a first (imaging) configuration,

FIG. 9 shows a side view of the head clamp when attached to a head and indexed into a first (imaging) configuration,

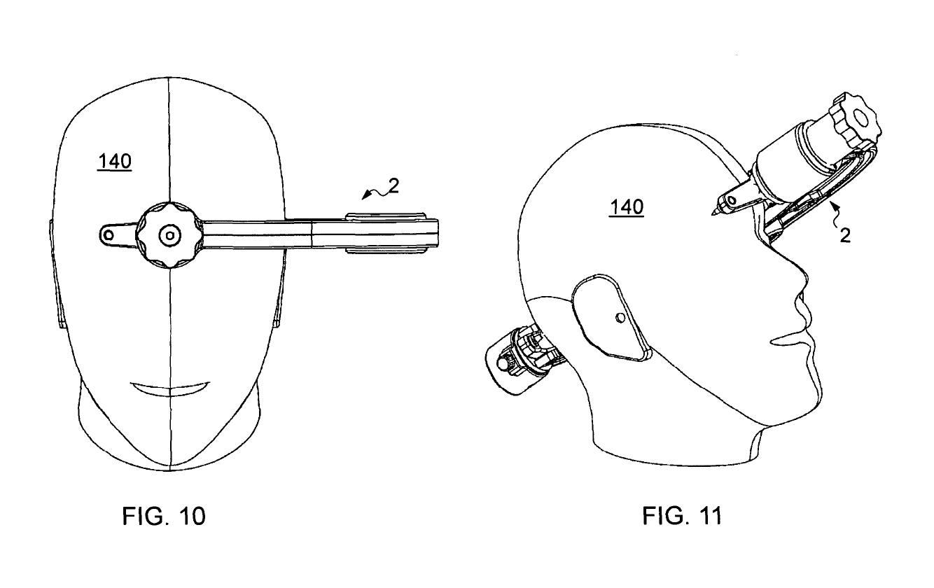

FIG. 10 shows a frontal view of the head clamp when attached to a head and indexed into a second (surgical) configuration,

FIG. 11 shows a side view of the head clamp when attached to a head and indexed into a second (surgical) configuration,

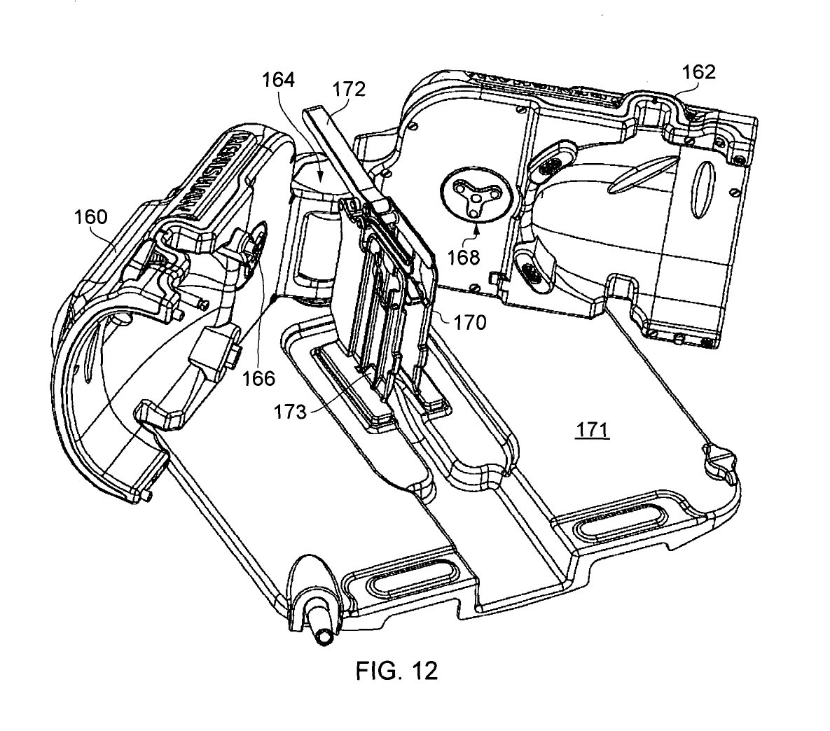

FIG. 12 shows MRI imaging apparatus for receiving a head clamp of the type shown in FIGS. 1 to 11 in an open configuration,

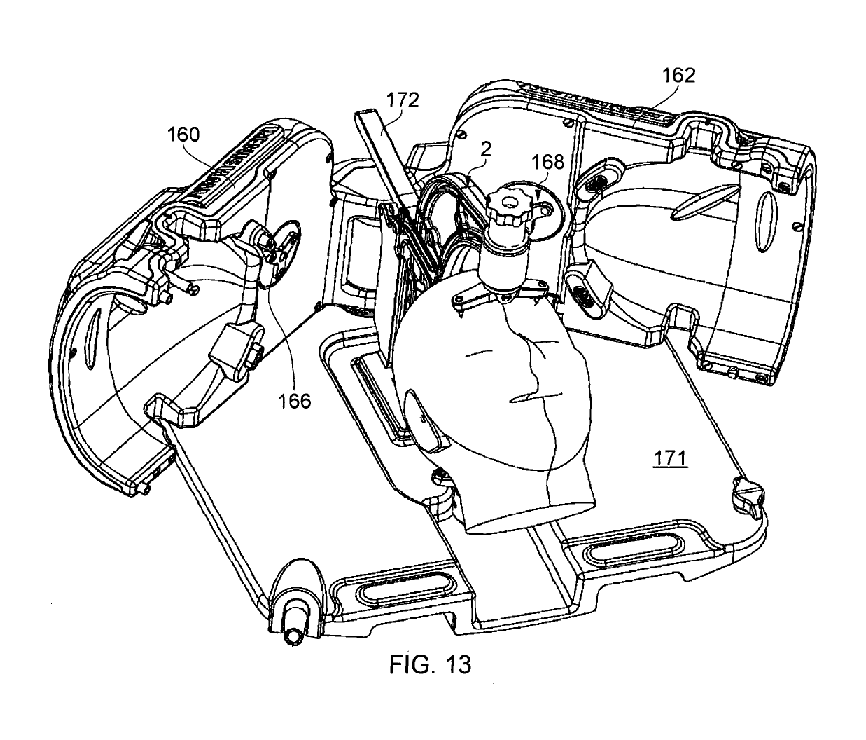

FIG. 13 shows a head clamp retained by the MRI apparatus,

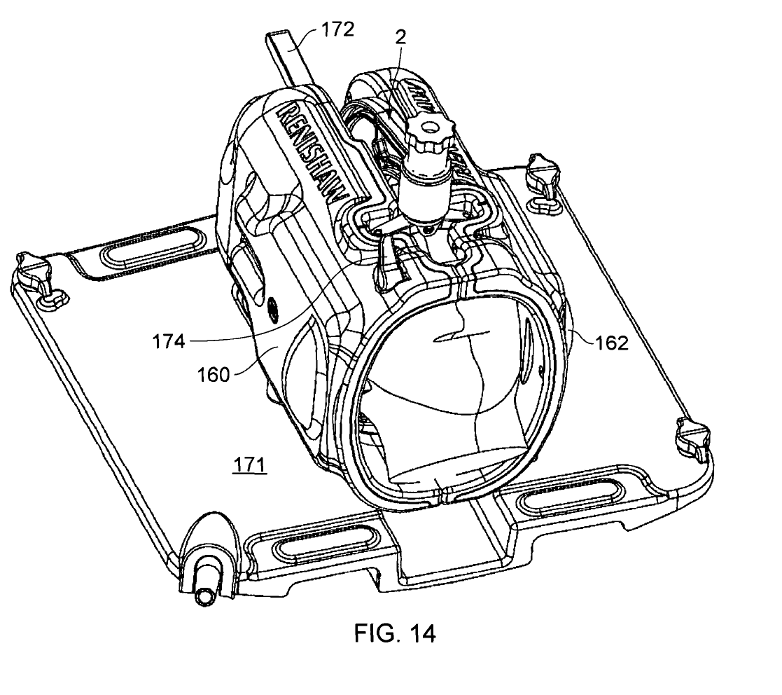

FIG. 14 shows the MRI imaging apparatus in a closed configuration,

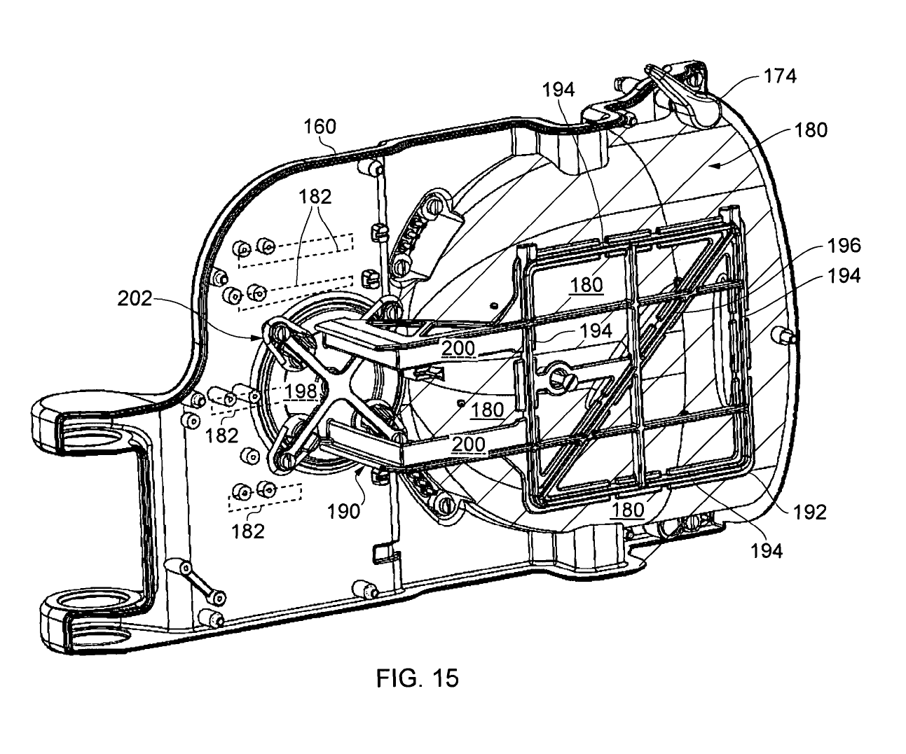

FIG. 15 shows one part of the housing of the MRI imaging apparatus in more detail,

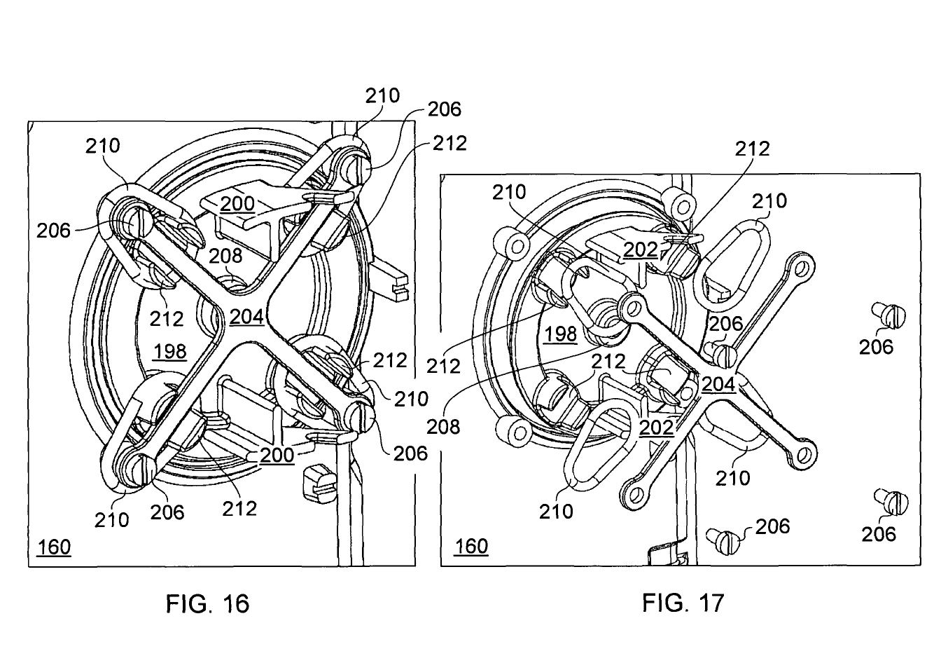

FIG. 16 provides a more detailed view of how the fiducial marker assembly is attached to the housing,

FIG. 17 provides an exploded view of how the fiducial marker assembly is attached to the housing,

FIG. 18 shows an optional support or back-up clamp for the head clamp,

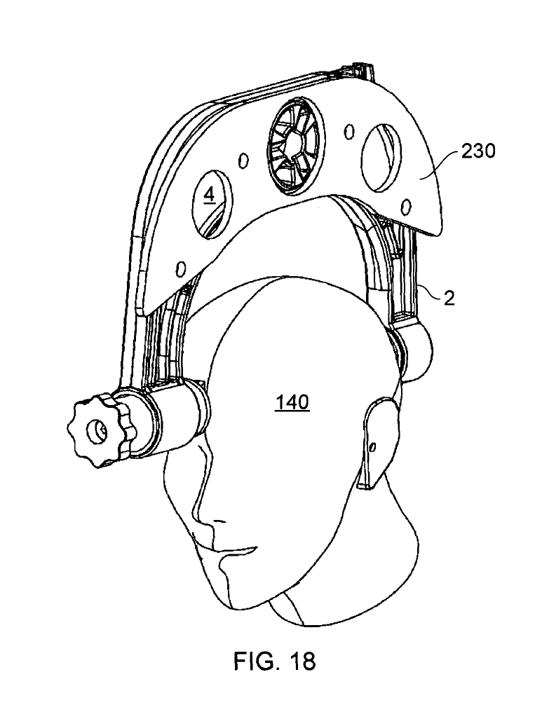

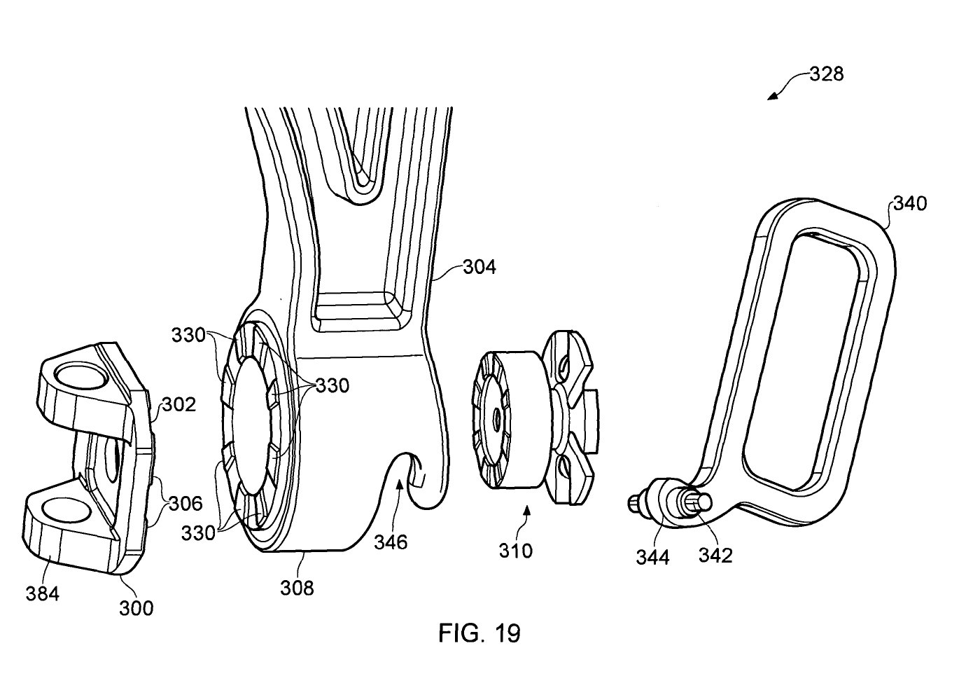

FIG. 19 is an exploded view of a Hirth coupling that can provide an alternative indexing mechanism for the head clamp of FIG. 1,

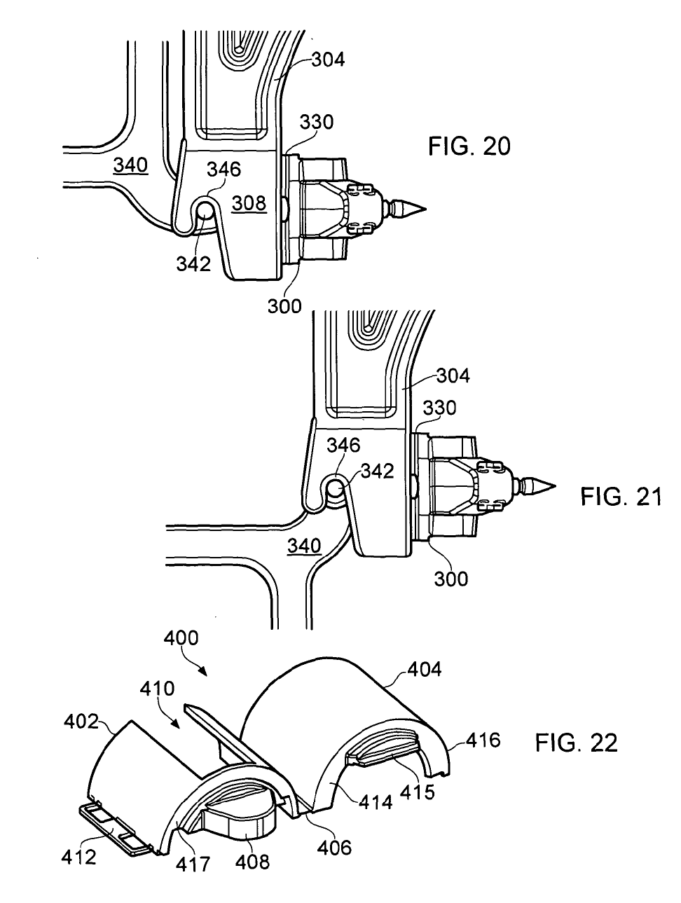

FIG. 20 shows the Hirth coupling of FIG. 19 in an unlocked configuration, FIG. 21 shows the Hirth coupling of FIG. 19 in a locked configuration,

FIG. 22 shows an anti-rotation device for locking the force applicator mechanism of a head clamp as shown in FIG. 1,

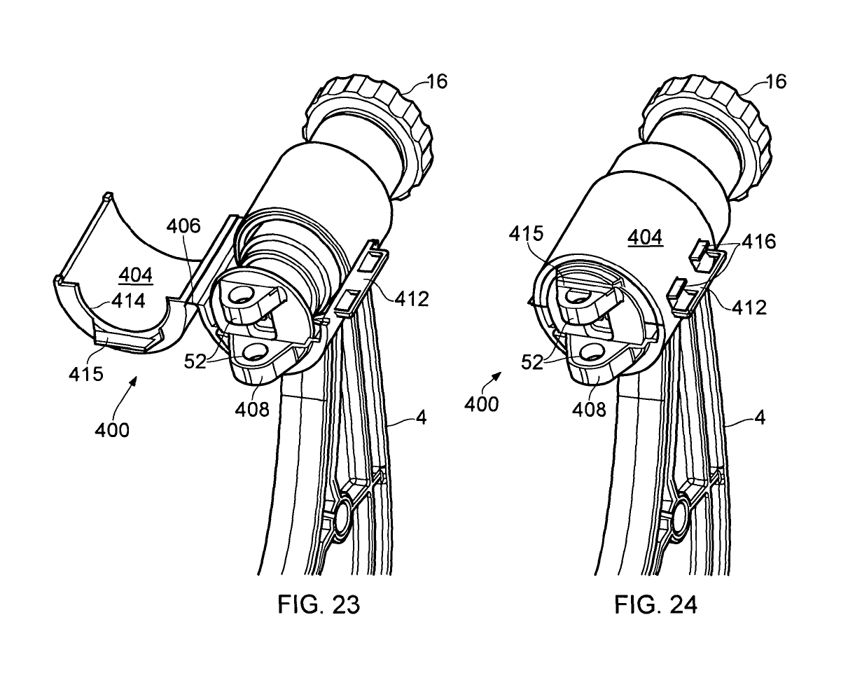

FIG. 23 shows the anti-rotation device of FIG. 22 in an open configuration when engaging the head clamp, and

FIG. 24 shows the anti-rotation device of FIG. 22 in a closed configuration that prevents rotation of the force applicator mechanism.

Referring to FIG. 1, a head clamp 2 of the present invention is illustrated. The head clamp 2 comprises a generally c-shaped member 4 having a first end 6 and a second end 8.

At the first end 6 of the c-shaped member 4, a first pair of skull attachment pins 10 and 12 are mounted to a first v-shaped pin carrying member 14. The first v-shaped member 14 is pivotally connected at its apex to a force applicator mechanism 16 which is in turn attached to the first end 6 of the c-shaped member 4. The first v-shaped member 14 is pivotable about the axis P1 such that the pins 10 and 12 can rotate in a single plane. The force applicator mechanism 16 comprises a profiled portion 18 which can be rotated to drive the first v-shaped pin carrying member 14 along the clamping force axis 20 thereby allowing the head clamp to be secured to the head of a subject. More details of the force applicator mechanism 16 are outlined below with reference to FIG. 2.

At the second end 8 of the c-shaped member 4 there is provided a second pair of skull attachment pins 22 and 24 that are mounted to a second v-shaped pin carrying member 26. The second v-shaped member 26 is pivotally connected to an indexing mechanism 28 that is in turn mounted to an aperture provided at the second end 8 of the c-shaped member 4. The second v-shaped member 26 is pivotable about the axis P2 such that the pins 22 and 24 can rotate in a single plane. The indexing mechanism 28 comprises a locking screw 29 for locking the selected indexed position. More details of the indexing mechanism 28 are provided below with reference to FIG. 3.

The c-shaped member 4 is approximately mechanically symmetrical about the plane 30 which provides a neutral axis of distortion. A neutral axis of distortion is thus the axis of symmetry of the c-shaped member 4 that is not subject to compression or tension (i.e. is not distorted) when the head clamp exerts a clamping force on a head. The c-shaped member 4 also comprises ribs 31 to improve stiffness and in particular to reduce the possibility of the c-shaped member 4 twisting. In this example, the c-shaped member 4 is formed from a glass-filled polymer material, such as Ryton R7, which is light, stiff and MRI compatible. It should be noted that it would also be possible to use other materials (e.g. ceramic) to form the c-shaped member. For example, a number of different polymer materials could be used and it would also be possible to use a ferrous metal (such as steel) if MRI compatibility was not required.

On both faces of the c-shaped member 4 there are provided identical datum features that lie on a neutral axis. The datum feature shown in FIG. 1 comprises three sub-features in the form of v-grooves 32a-32c that are radially spaced apart from one another by 120° and extend along directions that intersect at a common point. As explained in more detail below, the v-grooves 32a-32c of the datum feature provide a repeatable, kinematic, mechanical link with associated apparatus such as a stereoguide or MRI fiducial marker assemblies. The c-shaped member 4 also comprises an attachment feature on each face. The attachment feature is, in this example, provided in the form of three recesses 34a-34c and a circular rim portion 35.

The three recesses 34a-34c and/or the circular rim portion 35 can be used to provide a pseudo-kinematic link that allows the head clamp to be secured to apparatus such as a surgical bed or MRI apparatus in an approximate position. In this example, attachment features are provided on both faces that are co-axial with the datum features and therefore also lie substantially on the neutral axis. It would, of course, also be possible to space apart the attachment and datum features and/or provide either feature on only one face. In this example, supplementary attachment features are also provided by the protrusion 37 and the apertures 38 provided in the c-shaped frame 4. Apparatus may be secured to the head clamp 2 using some or all of the attachment features and supplementary attachment features as appropriate; this is described in more detail below.

More details of the various parts of the head clamp of FIG. 1 will now be described with reference to FIGS. 2 to 7.

Referring to FIG. 2, the force applicator mechanism 16 is illustrated in an exploded view. The force applicator mechanism 16 comprises a force control member 40 that comprises the profiled portion 18 at its proximal end, a force indication rod 42, a helical spring 44, a hollow cylindrical sleeve 48 that fits within the aperture at the first end 6 of the c-shaped member 4 and a shaft portion 50. An optional clamping element 46 may also be provided to lock the force control member 40 to the first end 6 of the c-shaped member 4. The distal end of the shaft portion 50 comprises a yoke 52 to which the v-shaped pin carrying member 14 can be attached using a pair of pivot bolts 56 and 58 and a threaded loading stud 54. Skull attachment pins 10 and 12 can be attached to the tapered apertures of the v-shaped pin carrying member 14.

The force applicator mechanism 16, when assembled, allows a predetermined force to be applied to the skull via the skull attachment pins 10 and 12. In particular, rotation of the force control member 40 causes the spring 44 to urge the shaft portion 50 along the clamping axis 20 thereby moving the pins 10 and 12 into engagement with the skull. As the clamping force applied to the skull is increased, the spring 44 compresses which in turn causes the force indication rod 42 to move relative to the force control member 40. The face 60 of the force indication rod 42 become flush with the face 62 of the profiled portion 18 when the required, preset, clamping force is being applied. Although not shown, an optional force limiter may be provided to prevent the preset clamping force being exceeded.

Referring now to FIG. 3, the tactile indicator of the force applicator mechanism 16 is shown before the predetermined clamping force is applied. It can be seen that the face 60 of the force indication rod 42 is sub-flush or recessed with respect to the face 62 of the profiled portion 18 of the force control member 40.

Referring to FIG. 4, the tactile indicator of the force applicator mechanism 16 is shown when the required, predetermined, clamping force is applied. It can be seen that the face 60 of the force indication rod 42 is flush with respect to the face 62 of the profiled portion 18 of the force control member 40. This provides the user with a simple, tactile, indication that the desired clamping force is being applied.

Referring to FIG. 5, the indexing mechanism 28 is shown in an exploded view with insets providing alternative views of several components. The indexing mechanism comprises a detent insert 70, an indexer body ring 72, a locking or indexing screw 29 and an indexer-head shaft 74. The distal end of the indexer-head shaft 74 comprises a yoke 84 to which the v-shaped pin carrying member 26 can be attached using a pair of pivot bolts 88 and 90 and threaded loading stud 86. Skull attachment pins 22 and 24 can be attached to tapered apertures of the shaped pin carrying member 26.

The indexer-body ring 72, when assembled, is moulded into an indexer-eye 73 provided at the inwardly facing side of the aperture provided at the second end 8 of the c-shaped member 4. The proximal end 76 of the indexer-head shaft 74 can, when the locking screw 29 is unscrewed, freely rotate within the indexer body ring 72. The indexing mechanism is configured to operate when the head frame is clamped to the head and hence, in use, there will be a force of around 400N urging the indexer-head shaft 74 into engagement with the indexer-body ring 72.

The locking screw 29 has a conical tip 79 and the indexer-head shaft 74 comprises a chamfered collar 80 in which are formed a plurality of conical depressions 75.

The indexer-body ring 72 comprises three chamfered buttresses. The required indexed position is locked when the conical tip 79 of the indexer screw 29 emerges through the hole 73 in the indexer-body ring 72 and engages with one of the conical depressions 75 of the indexer-head shaft 74. When the screw 29 is fully engaged with a conical depression 75, the indexer-head shaft 74 is displaced fractionally away from its rotation axis so that its chamfered collar 80 is pushed into and supported by two chamfered buttresses 78 of the indexer-body ring 72. This three point support provides backlash free and accurate positioning.

The detent insert 70 is a moulded thermoplastic component that is held in place in the outwardly facing side of the aperture provided at the second end 8 of the c-shaped member 4 by a pair of sprung lugs 71. The purpose of the detent insert 70 is to guide the indexer-head shaft 74 into angular positions relative to the indexer-body ring 72 that are close enough to the locked or indexed positions to enable the indexer screw 29 to engage the selected one of the conical depressions 75. The detent insert 70 also forms the function of providing tactile feedback to a user that an indexed position has been achieved by clicking into place. The detent insert 70 comprises four protruding sprung elements 73 that provide this positional click by engaging with complementary slots 81 located at the index positions that are provided at the proximal end 76 of the indexer-head shaft 74.

The indexer-head shaft 74 is made in a single piece. This is not essential but has been found to aid positioning between the indexing components (e.g. the chamfered collar 80 and conical depressions 75) and the yoke 84 whilst also simplifying assembly and cleaning. Assembly of the indexer-head shaft 74 and the indexer-body ring 72 is made possible by the inclusion of slots 82 in the chamfered collar 80 which can be aligned with and pass over the chamfered buttresses 78.

The indexing mechanism 28 thus allows a plurality of discrete and repeatable indexed positions to be provided. It should, however, be noted alternative indexing mechanisms could be used to provide the same function. For example, the indexing mechanism may be implemented using a v-tooth (e.g. Curvic or Hirth) coupling, a plurality of pins that mate with a plurality of tapered holes or complementary sets of balls. Instead of the indexing mechanism of the present example, different types of position setter could be provided; for example, a rotary position encoder could be provided instead of the indexing mechanism.

Referring to FIG. 6, a pin 100 for engaging the skull of a subject is shown. Such a pin may be used in apparatus as described above or as part of any other appropriate surgical apparatus. The pin 100 comprises a titanium core having a tapered portion 122 (e.g. that can be pushed into the tapered bore on the arm of the v-shaped member) and a sharp skull engaging tip 124. Although a tapered connection between the pin and member is shown, other connections (e.g. threaded connections) may be provided. The skull engaging tip 124 is coated with a ball of a soft, e.g. rubber, material 126. This soft coating or material 126 helps prevent unwanted damage to soft tissue (e.g. skin) whilst a head clamp is being positioned but it can be readily pierced by the titanium tip when the pins are located in the required position and a clamping force is applied. It should be noted that the soft coating, although preferred, is by no means essential. Similarly, the pins may be formed from many materials other than titanium (e.g. other metals or ceramic etc). The pin 100 may be permanently attached to a part of the head clamp but it is preferably provided as disposable (e.g. single use) item that can be easily attached to and detached from the head clamp.

Referring to FIG. 7, the datum and attachment features of the head clamp are shown in more detail. The datum feature comprises three v-grooves 32a-32c that are radially spaced apart from one another by 120°. The attachment feature comprises three recesses 34a-34c and the circular rim portion 35. Apertures 38 that are provided as part of the supplementary attachment features are also shown.

A number of potential uses of the head clamp described with reference to FIGS. 1 to 7, along with methods of attaching the head clamp to a subject, will now be described with reference to FIGS. 8 to 11.

In use, the head clamp 2 is placed over a subject's head so that the c-shaped member 4 partially encircles the head. The pins 10 and 12 provided at the first end 6 of the c-shaped member 4 are aligned with the centre of the forehead and the pins 22 and 24 provided at the second end 8 of the c-shaped member 4 are located on the opposite side (rear) of the head. The clamping axis 20 of the head clamp is also approximately aligned with the cantho-metal plane. An alignment aid, for example a band attached to the head or an alignment aid mounted to the head clamp, may be used to facilitate the required alignment. The first and second v-shaped members 14 and 26 are also respectively arranged such that the pins 10 and 12 pivot in substantially the same plane as the pins 22 and 24. Once aligned, the profiled portion 18 of the force applicator mechanism 16 is turned thereby advancing the pins 10 and 12 towards the head. The applied force is then increased until the pins 10, 12, 22 and 24 cut through the skin and engage the underlying skull bone with the required, predetermined, amount of force. In this example, a total force of 400N is applied which results in each pin applying a 200N force to the skull bone. Pivoting of the first and second v-shaped members 14 and 26 ensures that the force is evenly applied to the skull through each pin.

Applying a predetermined (preset) amount of force evenly distributed through the pins has the advantage that the c-shaped member 4 will deform by substantially the same amount each time it is attached to a head. Furthermore, locating the datum and attachment features substantially on the neutral axis ensures that the position of such features is substantially invariant even if the c-shaped member is subjected to slightly different distortion forces.

FIGS. 8 and 9 show the head clamp 2 attached to a head 140 and indexed into a first, imaging, position. In this first position, the c-shaped member extends around the top of the head 140. This position is particularly suited for imaging applications, such as MRI, because it allows MRI coils and/or fiducial markers to be placed in close proximity to both sides of the head without any interference from the c-shaped member 4.

FIGS. 10 and 11 shows the head clamp 2 attached to the head 140 and indexed into a second position in which the c-shaped member extends around the side of the head. This second position provides the access to the top of the head that a surgeon typically requires to perform stereotactic neurosurgery.

Although two different indexed positions are shown in FIGS. 8-11, it should be noted that the c-shaped member of the head clamp could be indexable between more positions. For example, the c-shaped member could also be indexable into a position on the other side of the head and/or into any one or more other (e.g. intermediate) indexed positions. It is, however, preferably to provide only a few index positions to ensure that there is no potential for confusion over the index position that has been set. Markings may also be provided, if required, to help indicate the indexed position that has been adopted.

The positional differences between the various indexed positions adopted by the head clamp are preferably known or measured. For example, the head clamp may be designed so that there is a predetermined positional change in the position of one or more datum features between each of the indexed positions. Alternatively, a calibration procedure may be performed prior to use (e.g. during manufacture of the head clamp) in which the position of the datum feature is measured for different indexed positions. The head clamp may thus be supplied with a set of coordinate transformations that provide such position mapping information.

It should be noted that the head clamp may be formed from the glass filled polymer material mentioned above or any other suitable alternative material(s). The various parts of the head clamp may be single use (disposable), multi-use or re-useable. If any part of the head clamp can be used more than once, it is preferred that such a part can be sterilised in an autoclave.

Referring now to FIGS. 12 to 17, there will be described apparatus for imaging the head of subject that is designed for use with a head clamp of the type described with reference to FIGS. 1 to 11. Although the apparatus may be used with any imaging technique, the following examples describe its use with MRI apparatus.

FIG. 12 illustrates head imaging apparatus suitable for use in MRI that is placed in its open position. The apparatus comprises a housing formed from a first housing part 160 that is connected to a second housing part 162 by a pivot joint 164. As will be described in more detail with reference to FIG. 14-16 below, each housing part comprises a hollow plastic shell that contains RF coil assemblies for MRI, various electronic control circuitry and a floating fiducial marker assembly. The fiducial marker assemblies of the first and second housing parts are retained in their respective housing parts and have inwardly facing datum features 166 and 168 that are externally accessible through an aperture formed in the housing. A clamp mechanism in the form of a toggle clamp 170 is provided on the base 171 of the apparatus between the two housing parts 160 and 162.

As illustrated in FIG. 13, the toggle clamp 170 is designed such that activation of lever 172 causes the clamp to engage and hold the circular rim portions 35 of the two attachment features provided on the opposed faces of the c-shaped member 4 of the head clamp 2 that is described above with reference to FIGS. 1 to 11. The head imaging apparatus also comprises a channel 173 having angled walls for engaging the protrusion 37 of the c-shaped member 4 to provide additional mechanical support. The toggle clamp 170 is also arranged such that, when engaging the circular rim portions 35 of the head clamp 2, the datum features of the head clamp 2 are still accessible. In particular, the datum features 166 and 168 of the fiducial marker assemblies are arranged such that they can be brought into contact with the complementary datum features of the head clamp when held by the toggle clamp 170.

FIG. 14 illustrates the apparatus described above with reference to FIGS. 12 and 13 in its closed position. A two-part locking latch 174 is provided to hold the housing parts together in the closed position. In this closed position, the datum features 166 and 168 of the fiducial marker assemblies are biased into engagement with the complementary datum features of the head clamp 2. The fiducial markers of the fiducial marker assemblies are thus accurately held in a known, repeatable, position relative to the datum features of the head clamp. These fiducial markers thus act as highly accurate and repeatable reference position markers in any acquired MRI images.

Referring to FIG. 15, the components located within the first housing part 160 of the apparatus described with reference to FIGS. 12 to 14 will be described. It should be noted that the second housing part 162 houses similar components and is therefore not shown.

The first housing part 160 comprises a plastic shell that holds the various sets of

RF coils 180 that can be used in conjunction with MRI apparatus to obtain high resolution images of the head. These RF coils 180 are secured to the housing and connected to electronic control circuitry provided on circuit boards 182. The RF coils 180 and circuitry 182 typically handle electrical signals during use and the first housing part therefore also acts as an insulating shield that prevents patients and operators being exposed to the electrical voltages. Cables to and from the electrical circuitry 182 are routed via the pivot joint of the housing.

The first housing part 160 also contains a first floating fiducial marker assembly 190. The fiducial marker assembly 190 comprises a fiducial marker 192 in the form of a square frame 194 with a diagonal cross member 196. The fiducial marker 192 comprises or contains a material that is MRI visible, such as copper sulphate solution. The fiducial marker assembly 190 also comprises a circular datum portion 198 having a surface protruding through an aperture in the housing that provides the externally accessible datum feature 166. A rigid right angled framework section 200 is also provided to connect the datum feature 166 to the fiducial marker 192.

As will be explained in more detail below, the fiducial marker assembly 190 is retained within the housing part 160 but is free to move relative to that housing part (i.e. it can be said to be floating or substantially unconstrained). This prevents any distortions of the housing being passed to the fiducial marker assembly and also permits the datum feature of the fiducial marker assembly to always adopt the same position relative to the complementary datum feature of the head clamp even if the housing parts do not adopt repeatable relative positions. A biasing mechanism 202 is, however, provided to retain the fiducial marker assembly and to bias the datum feature of that assembly into engagement with the head clamp during use.

Referring to FIGS. 16 and 17, the biasing mechanism 202 of the first housing part 160 described above is shown in cut-away and exploded cut-away views respectively.

The biasing mechanism 202 comprises an x-shaped flexible member 204 having four legs with ends that are each secured to the housing part 160 by separate screws 206. The centre of the x-shaped flexible member 204 engages a spherical protrusion 208 provided at the centre of the circular datum portion 198 of the fiducial marker assembly 190; the spherical protrusion 208 thus provides a single point of contact with the substantially flat flexible member 204. The biasing mechanism 202 also comprises four flexible loops 210. Each loop 210 is captured between the housing part 160 and one of the legs of the x-shaped flexible member 204 and also engages a protruding feature 212 provided on the circular datum portion 198. The loops are held in slight tension and, because they are equally spaced around the circumference of the circular datum portion 198, they maintain the circular datum portion 198 in a substantially central position within the aperture of the housing. The flexibility of the loops 210 and x-shaped member 204 does, however, allow movement of the fiducial marker assembly in all 6 degrees of freedom when necessary so as to enable the datum feature 166 to adopt the necessary position relative to a complementary datum feature provided on a head clamp. It should also be noted that the biasing mechanism 202 provides the only mechanical connection between the housing and the fiducial marker assembly and consequently any distortions of the housing will not be transmitted to that assembly.

The head clamp and imaging apparatus described above are designed to be used together, but it is important to note that each could be used separately for different purposes. For example, the head clamp could be used purely for clamping a head during surgical or other procedures. Similarly, the imaging apparatus could be arranged to engage and image other body parts to which other fixtures are attached.

It should be remembered that the head clamp described above with reference to FIGS. 1 to 11 is also designed to be used in surgical procedures. In particular, once an MRI image of the head has been obtained, the head clamp may be indexed to at least one further position that is suitable for conducting a surgical procedure.

The, or each, datum feature provided on the head clamp may thus be used to locate other apparatus in a known position relative to the head clamp. For example, a datum feature could be used to accurately position, relative to the head clamp, retro-reflective surgical navigation instruments, surgical robots such as the Renishaw-Mayfield neuromate (Registered Trade mark) robot, and targeted radiotherapy devices such as the Leksell gamma-knife (Registered Trade mark) apparatus. Position information acquired from the MRI images can then be tied back to the datum feature on the head clamp and can therefore be used to precisely target regions or points in the brain.

Although the mechanical strength of the c-shaped member 4 of the above described head clamp is designed to be more than adequate for all expected mechanical loads to which it will be subjected in use, it is noted that the head clamp may, on very rare occasions, be subjected to large mechanical impulses that could exceed safe design limits. For example, a surgeon may have no choice other than subjecting the head clamp to large forces to perform an emergency procedure (e.g. resuscitation etc). In the case of a c-shaped member formed from a rigid material, such as a glass filled polymer, any failure may be catastrophic; this can obviously have severe consequences if a neurosurgical procedure is in progress.

Referring to FIG. 18, a supplementary support or backup clamp 230 (e.g. formed from metal) is shown that can be attached to the head clamp 2 during surgery. The backup clamp 230 may, for example, attach to the apertures 38 (not visible in FIG. 18 but shown in FIG. 1) formed in the c-shaped member 4 of the head clamp 2. The supplementary clamp 230 is arranged so that access to the datum feature of the head clamp 2 is maintained. In normal use, the supplementary clamp 230 takes none of the mechanical load of the head clamp 2 but it does, however, act as a backup device that takes the full mechanical load of the head clamp 2 if that head clamp 2 was to fail. The supplementary clamp 230, which is by no means essential, thus mitigates the unwanted consequences that might occur with a catastrophic failure of the head clamp 2.

Referring to FIG. 19, an exploded view is shown of an indexing mechanism 328 suitable for inclusion in a head clamp as described above. The indexing mechanism 328 may, for example, be provided instead of the indexing mechanism 28 described in detail with reference to FIG. 5.

The indexing mechanism 328 comprises a so-called Hirth coupling in which a series of concentric features 330 are provided around the aperture at the second end 308 of the c-shaped member 304. An indexable part 300 is also provided that has a face plate 302 comprising a plurality of concentric features 306 that complement the concentric features 330 of the c-shaped member 304. In particular, the complementary features 330 and 306 are configured such that, when biased into engagement, the indexable part 300 can adopt (i.e. can be indexed into) any one of multiple different orientations relative to the c-shaped member 304.

An indexing adjuster 310 and release lever 340 are also provided. The indexing adjuster 310 is insertable into the aperture at the second end 308 of the c-shaped member 304 and includes flexural elements to provide a preload bias (i.e. to bias the complementary features 330 and 306 into engagement even when the head clamp is not loaded) and stops to prevent the part being overloaded. The associated release lever 340 comprises a cam 344 and an axle 342. The axle 342 can be attached (clipped into) corresponding slots 346 provided at the second end 308 of the c-shaped member 304.

Rotation of the release lever 340, when the axle 342 is clipped into the slots 346, allows the cam 344 to engage and disengage the back surface of the indexing adjuster 310. Rotation of the release lever 340 can thus be used to force the cam 344 against the back surface of the indexing adjuster 310 to separate the complementary features 330 and 306. Once these features are separated, the indexable part 300 can be indexed into a different orientation and locked in that orientation by rotating the release lever 340 to reduce the force applied by the cam 344. In other words, the indexing adjuster 310 and release lever 340 allow a user to axially separate the complementary features 330 and 306 to enable the indexable part 300 to be rotated into a different orientation relative to the second end 308 of the c-shaped member 304.

The indexable part 300 also comprises a yoke 384 to which a v-shaped pin carrying member (not shown in FIG. 19 but included in FIGS. 20 and 21 below) can be pivotally mounted in a similar manner to that described above with reference to FIG. 5.

Referring next to FIG. 20, the indexing mechanism 328 of FIG. 19 is shown when assembled. FIG. 20 shows the release lever 340 in the vertical or unlocked orientation in which the cam 344 and indexing adjuster 310 (not visible in FIG. 20) have forced the indexable part 300 out of engagement with the features 330 provided at the second end 308 of the c-shaped member 304. In this unlocked configuration, the c-shaped member 304 may be indexed into the required position.

Referring to FIG. 21, the indexing mechanism 328 of FIG. 19 is again shown when assembled but with the release lever 340 in the horizontal or locked orientation in which its cam 344 (not visible in FIG. 21) does not apply a disengaging force via the indexing adjuster 310 (also not-visible in FIG. 21). The features of the indexable part 300 thus engage the features 330 provided at the second end 308 of the c-shaped member 304. In this locked configuration, the c-shaped member 304 is securely held in the required indexed position.

A Hirth coupling of this type has the advantage of providing accurate metrology whilst allowing multiple, repeatable, angular orientations to be selected. In particular, the arrangement does not necessarily require a separate locking piece because of the angle-defining features 330 and 306 provided at the second end 308 of the c-shaped member 304 and the face plate 302 respectively. These angle-defining features may, for example, be formed (e.g. ground) in the frame and/or face plate or may be formed in a component that is subsequently over-moulded into the end of the frame or into the face plate. The Hirth coupling offers low levels of backlash and self-centres. A further benefit of the Hirth coupling is that it is self-locking and resistant to accidental release when under load; the dimensions of the interlocking elements are also selected such that the mechanism can only engage in the allowed orientations (e.g. 0° and ±90°. Preferably, all of the components of the indexing mechanism 328 are MRI benign.

A head clamp of the type described above comprises a c-shaped member having an indexing mechanism at one end and a force applicator mechanism at the other end. Each of these mechanisms preferably carry one or more pins for engaging the skull of a subject. Although these pins may be coated with a soft material, they can still present a sharps risk to the patient when the head clamp is being attached or removed.

The indexing mechanism of the head clamp is preferably lockable in various indexed positions when not loaded (i.e. when not attached to the skull of a subject). The various skull attachment pins carried by the indexing mechanism are thus prevented from rotating about the clamping axis when the head clamp is being attached or removed. In contrast, the pins carried by the force applicator mechanism are typically free to rotate about the clamping axis until they engage the skull. This free rotation can, in some instances, pose a sharps risk to the patient who may have their forehead scored from a rotating yoke and associated skull mounting pins during fitting or removal of the head clamp.

Referring to FIGS. 22 to 24, a yoke hold or anti-rotation device 400 is illustrated for reducing the risk of skin injury resulting from the free rotation of skull attachment pins that may be permitted by the force applicator mechanism.

FIG. 22 shows the anti-rotation device 400 in an open configuration. The anti-rotation device 400 comprises a first portion 402 linked to a second portion 404 by a hinge 406. The first and second portions 402 and 404 have a generally half cylindrical shape. The first portion 402 comprises a cradle grab 408, a slot 410, a clasp 412 and a circumferential boss 417 . The second portion 404 comprises a circumferential boss 414, a protrusion 415 and a pair of ledges 416 (only partially visible in FIG. 20) that can mate with the clasp 412 of the first portion. The anti-rotation device 400 may be formed as a single piece from, for example, a suitable polymer. The anti-rotation device 400 may be a single use item or a multi-use item that is suitable for sterilisation.

As shown in FIGS. 23 and 24, the anti-rotation device 400 shown in FIG. 22 may be attached to the force applicator mechanism 16 described above with reference to FIGS. 1 to 4.

FIG. 23 shows the anti-rotation device 400 in its open configuration with the first portion 402 placed into contact with the force applicator mechanism 16.

FIG. 24 shows the anti-rotation device 400 after it has been closed around the force applicator mechanism 16. The pair of ledges 416 and clasp 412 cooperate to provide a snap fit lock that retains the anti-rotation device 400 in position. The slot 410 receives the c-shaped member 4 and prevents rotation of the anti-rotation device 400 relative to that c-shaped member 4. The cradle grab 408 and protrusion 415 of the anti-rotation device 400 engage the yoke 52 of the force applicator mechanism 16 and prevent it from rotating. In this manner, the yoke 52 is locked in a single orientation relative to the c-shaped member 4.

It should be noted that the anti-rotation device 400 can travel back and forth along the clamping axis with the yoke 52. In other words, the anti-rotation device 400 does not interfere with the normal operation of the force applicator mechanism 16 (i.e. which drives the yoke 52 back and forth). The anti-rotation device 400 can thus be used when attaching and/or detaching the head clamp to a subject. Removal of the anti-rotation device 400 (e.g. by unclipping it) when the head clamp is secured to a subject permits the c-shaped member to be indexed into the required orientation(s). In this manner, the anti-rotation device 400 reduces the sharps risk associated with using the head clamp without an adverse effect on its function.

It should be noted that the anti-rotation device 400 is by no means essential for operation of the head clamp. It should also be noted that alternative means for preventing rotation of the yoke carrying the skull attachment pins may be provided. For example, the force applicator mechanism 16 may comprise an integral anti-rotation component. Although the anti-rotation device 400 is described for use with the force applicator mechanism 16, the skilled person would also appreciate a similar device may be used to prevent any unwanted rotation of other force applicator mechanisms and/or the indexing mechanism. For example, a similar anti-rotation device may be used for alternative indexing mechanisms that have a freely rotatable yoke prior to engagement of the head clamp with the skull.

It should again be noted that the above embodiments are merely examples of the present invention. The skilled person would be aware of the many variations and alternative embodiments that would be possible.

1. Apparatus for imaging a body part of a subject, comprising; a housing for at least partially surrounding a body part, and a first fiducial marker assembly retained at least partially within the housing that comprises one or more fiducial markers and a datum feature, the position of the datum feature being fixed relative to the one or more fiducial markers, wherein the first fiducial marker assembly is moveable with respect to the housing and the datum feature is accessible from outside of the housing.

2. An apparatus according to claim 1, wherein the housing defines an internal cavity and one or more RF coils for magnetic resonance imaging are provided within the internal cavity.

3. An apparatus according to claim 1, wherein the one or more fiducial markers can be imaged using magnetic resonance imaging.

4. An apparatus according to claim 1, wherein the portion of the first fiducial marker assembly that comprises the datum feature protrudes from the housing through an aperture.

5. An apparatus according to claim 1, wherein the housing comprises a first housing part and a second housing part, wherein the first and second housing parts can be moved into a closed position that defines an imaging space in which a human head can be located.

6. An apparatus according to claim 5, wherein the first housing part is attached to the second housing part via a pivot joint, wherein the first and second housing parts can be pivoted into the closed position.

7. An apparatus according to claim 6, wherein one or more electrical cables are routed to and/or from the first and second housing parts through the pivot joint.

8. An apparatus according to claim 5, comprising a second fiducial marker assembly comprising one or more fiducial markers located within the housing that have a fixed position relative to a datum feature that is accessible from outside of the housing, wherein the first housing part contains the first fiducial marker assembly and the second housing part contains the second fiducial marker assembly.

9. An apparatus according to claim 8 for receiving a head clamp that is attached to the head of a subject, wherein the datum features of the first and second fiducial marker assemblies are brought into contact with complementary datum features provided on the head clamp when the first and second parts of the housing are moved into the closed position.

10. An apparatus according to claim 1, comprising a clamp mechanism for releasably retaining a fixture that is attached to the body part of a subject.

11. An apparatus according to claim 1, wherein the datum feature comprises a kinematic datum feature.

12. A kit comprising apparatus according to claim 1 and a fixture attachable to a body part, wherein the fixture comprises at least one datum feature that is complementary to the datum feature of the apparatus thereby allowing the first fiducial marker assembly to be repeatably located in a predetermined position relative to the fixture.

13. A kit according to claim 12, wherein the fixture comprises a head clamp.

14. A kit according to claim 13, wherein the head clamp comprises a c-shaped member for partially encircling the head of a subject, and first and second skull attachment portions provided at first and second ends of the c-shaped member, wherein the head clamp comprises an indexing mechanism that allows the c-shaped member to be indexed between at least two repeatable relative positions.

15. A kit according to claim 13, wherein the head clamp comprises a c-shaped member for partially encircling the head of a subject, and first and second skull attachment portions provided at first and second ends of the c-shaped member, wherein the c-shaped member comprises the at least one datum feature, wherein the at least one datum feature is located substantially on the neutral axis of distortion of the c-shaped member.
